(19) United States
(12) Patent Application Publication
(10) Pub. No.: US 2008/0131389 A1
(43) Pub. Date: Jun. 5, 2008

(54) Hair Relaxer

(75) Inventors: Akira Shibuya, Kawasaki-shi (JP); Makoto Saito, Kawasaki-shi (JP)

Correspondence Address: SUGHRUE MION, PLLC, 2100 PENNSYLVANIA AVENUE, N.W., SUITE 800, WASHINGTON, DC, 20037, US

(73) Assignee: Showa Denko K.K., Minato-ku (JP)

(21) Appl. No.: 11/793,798

(22) PCT Filed: Feb. 3, 2006
(86) PCT No.: PCT/JP06/02301

§ 371 Date: Jun. 21, 2007

Related U.S. Application Data
(60) Provisional application No. 60/652,845, filed on Feb. 15, 2005.

(30) Foreign Application Priority Data
Feb. 7, 2005 (JP) 2005-030623

(51) Int. Cl. A61K 8/46; A61Q 5/04

(52) U.S. Cl. 424/70.5

(57) ABSTRACT

A hair relaxer includes at least one specific mercapto compound such as 2-methoxyethyl 3-mercaptopropionate, 2-mercapto-4-butanolide and 2-mercapto-4-butyrolactam. The hair relaxer can achieve an excellent effect of shaping and relaxing hair over a wide range of pH values from weak acidity to weak alkalinity. In particular, the hair relaxer displays a more superior hair shaping and relaxing performance in a weakly acidic to neutral pH range. Consequently, the hair relaxer can drastically reduce the damage to the hair and skin and can reliably perform straightening, uncurling, curling and other hair processing.

### CROSS REFERENCE TO RELATED APPLICATION

This application is an application filed under 35 U.S.C. §111(a) claiming benefit pursuant to 35 U.S.C. §119(e) of the filing dates of Provisional Application No. 60/652,845 filed Feb. 15, 2005, pursuant to 35 U.S.C. §111(b).

### FIELD OF THE INVENTION

The present invention relates to a hair relaxer that can work in a weakly acidic to neutral range and is best suited for the shaping and relaxing of hair such as straightening, uncurling and curling.

### BACKGROUND OF THE INVENTION

Hair processing agents containing sulfites and bisulfites have been widely used in the shaping and relaxing of hair such as straightening, uncurling and curling. The sulfites and bisulfites are also used in permanent waving agents. The hair processing agents for shaping and relaxing contain the sulfites or bisulfites at lower concentrations than for permanent waving and are used under very mild conditions. Consequently, the effects of shaping and relaxing hair are insufficient.

Furthermore, the sulfites are easily decomposed under acidic conditions, and therefore the hair processing agents containing the sulfites are generally alkaline. However, the alkalinity leads to problems such as hair damage and skin irritations.

To solve such problems, there have been proposed a hair processing composition containing sulfite or bisulfite, and imidazolidinedione (Patent Document 1) and a hair processing composition containing sulfite or bisulfite, and urea and alcohol (Patent Document 2).

Although Patent Document 1 explicitly describes that the composition with imidazolidinedione may be used on an acidic side by containing an acetate buffer solution, the improvement in hair shaping and relaxing effects is not such significant.

Patent Document 2 describes that the composition with urea and alcohol is used on an alkaline side in order to prevent decomposition of sulfite or bisulfite and consequently has the problem of hair damage due to alkalinity.

### [Patent Document 1] JP-A-S58-170710

### [Patent Document 2] JP-A-2000-229819

### DISCLOSURE OF THE INVENTION

It is an object of the present invention to provide a hair relaxer that can work in a weakly acidic to neutral range which is gentle to the skin and hair. More specifically, the invention has an object of providing a hair relaxer best suited for the shaping and relaxing of hair such as straightening, uncurling and curling.

The present inventors studied diligently in view of the background art as described above, and have found that an agent containing a specific mercapto compound can effectively produce an effect of shaping and relaxing hair at pH levels at which satisfactory results have not been achieved historically. The present invention has been completed based on the finding.

The present invention is concerned with the following [1] to [23].

[1] A hair relaxer comprising at least one mercapto compound represented by the following formula (1) or (2):

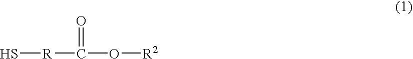

wherein R is an alkylene group of 1 to 6 total carbon atoms that may have a branch; R² is an alkoxyalkyl group of 3 to 15 total carbon atoms; and an alkylene part in R² may contain an ether linkage;

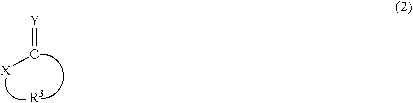

wherein X is a structure selected from the group consisting of a single bond, —O—, —S—, —NH— and —NR⁴—; R⁴ is an alkyl group of 1 to 6 carbon atoms; Y is an oxygen atom or a sulfur atom; and R³ is a divalent organic residue having at least one mercapto group.

[2] The hair relaxer as described in [1], wherein the mercapto compound represented by the formula (1) is a compound represented by the following formula (1a):

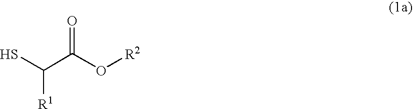

wherein R¹ is a hydrogen atom or an alkyl group of 1 to 5 carbon atoms; and R² is as defined in the formula (1).

[3] The hair relaxer as described in [2], wherein R¹ in the formula (1a) is a hydrogen atom or a methyl group.

[4] The hair relaxer as described in [2] or [3], wherein R² in the formula (1a) is a group selected from the group consisting of 2-methoxyethyl, 2-ethoxyethyl, 2-methoxypropyl, 2-ethoxypropyl, 1-methoxypropane-2-yl, 1-ethoxypropane-2-yl, 5-methoxy-3-oxapentyl and 5-ethoxy-3-oxapentyl.

[5] The hair relaxer as described in [2], wherein the mercapto compound represented by the formula (1a) is a compound selected from the group consisting of 2-methoxyethyl thioglycolate, 2-ethoxyethyl thioglycolate, 2-methoxyethyl thiolactate and 2-ethoxyethyl thiolactate.

[6] The hair relaxer as described in [1], wherein the mercapto compound represented by the formula (1) is a compound represented by the following formula (1b):

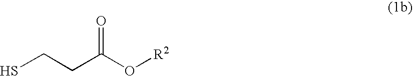

wherein R² is as defined in the formula (1).

[7] The hair relaxer as described in [6], wherein R² in the formula (1b) is a group selected from the group consisting of 2-methoxyethyl, 2-ethoxyethyl, 2-methoxypropyl, 2-ethoxypropyl, 1-methoxypropane-2-yl, 1-ethoxypropane-2-yl, 5-methoxy-3-oxapentyl and 5-ethoxy-3-oxapentyl.

[8] The hair relaxer as described in [6], wherein the mercapto compound represented by the formula (1b) is a compound selected from the group consisting of 2-methoxyethyl 3-mercaptopropionate and 2-ethoxyethyl 3-mercaptopropionate.

[9] The hair relaxer as described in [1], wherein X in the formula (2) is a structure selected from the group consisting of —O—, —NH—, —S— and —N(CH₃)—.

[10] The hair relaxer as described in [1], wherein X in the formula (2) is a single bond.

[11] The hair relaxer as described in any one of [1], [9] and [10], wherein Y in the formula (2) is an oxygen atom.

[12] The hair relaxer as described in any one of [1] and [9] to [11], wherein R³ in the formula (2) is an alkylene group having at least one mercapto group.

[13] The hair relaxer as described in any one of [1], [9] and [10], wherein when Y and R³ in the formula (2) are an oxygen atom and an alkylene group having at least one mercapto group, respectively, the mercapto group of R³ is bonded at the α-position of the carbonyl group.

[14] The hair relaxer as described in [1], wherein the mercapto compound represented by the formula (2) is a compound selected from the group consisting of 2-mercapto-4-butanolide, 2-mercapto-4-methyl-4-butanolide, 2-mercapto-4-ethyl-4-butanolide, 2-mercapto-4-butyrolactam, N-methyl-2-mercapto-4-butyrolactam, 2-mercapto-5-valerolactam, N-methyl-2-mercapto-5-valerolactam and 2-mercapto-6-hexanolactam.

[15] The hair relaxer as described in [1], wherein the mercapto compound represented by the formula (2) is a compound selected from the group consisting of 2-mercaptocyclopentanone and 2-mercaptocyclohexanone.

[16] The hair relaxer as described in any one of [1] to [15], wherein the hair relaxer contains the mercapto compound in an amount of 0.1 to 10% by mass.

[17] The hair relaxer as described in any one of [1] to [16], wherein the pH is in the range of 4.0 to 7.5.

[18] A shampoo comprising the hair relaxer as described in any one of [1] to [17].

[19] A rinsing conditioner comprising the hair relaxer as described in any one of [1] to [17].

[20] A conditioner comprising the hair relaxer as described in any one of [1] to [17].

[21] A hair treatment comprising the hair relaxer as described in any one of [1] to [17].

[22] A hair lotion comprising the hair relaxer as described in any one of [1] to [17].

[23] A hair mousse comprising the hair relaxer as described in any one of [1] to [17].

The hair relaxer according to the present invention can achieve an excellent effect of shaping and relaxing hair over a wide range of pH values from weak acidity to weak alkalinity. In particular, the hair relaxer displays a more superior hair shaping and relaxing performance in a weakly acidic to neutral pH range. Consequently, the hair relaxer according to the invention can drastically reduce the damage to the hair and skin and can reliably perform straightening, uncurling, curling and other hair processing. Therefore, the hair relaxer is very useful for the hair shaping and relaxing in which hair such as frizzy hair and curled hair is shaped and relaxed.

### PREFERRED EMBODIMENTS OF THE INVENTION

The present invention will be described in detail hereinbelow.

The hair relaxer according to the present invention includes at least one mercapto compound represented by the following formula (1) or (2):

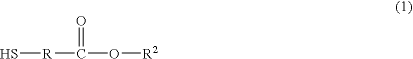

wherein R is an alkylene group of 1 to 6 total carbon atoms that may have a branch; R² is an alkoxyalkyl group of 3 to 15 total carbon atoms; and the alkylene part in R² may contain an ether linkage;

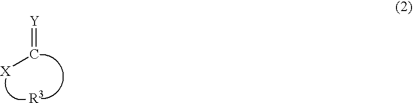

wherein X is a structure selected from the group consisting of a single bond, —O—, —S—, —NH— and —NR⁴—; R⁴ is an alkyl group of 1 to 6 carbon atoms; Y is an oxygen atom or a sulfur atom; and R³ is a divalent organic residue having at least one mercapto group.

The mercapto compounds will be described first.

The mercapto compounds employable in the hair relaxer of the invention are represented by the above-described formula (1) and/or (2).

In the formula (1), R is an alkylene group of 1 to 6 total carbon atoms that may have a branch, with examples including methylene, ethylene, methylmethylene, dimethylmethylene, ethylmethylene, propylmethylene, butylmethylene, methylethylene, dimethylethylene, 1,2-dimethylethylene, propylene, tetramethylene, pentamethylene and hexamethylene.

R² is an alkoxyalkyl group of 3 to 15 total carbon atoms, and the alkylene part in R² may contain an ether linkage. In view of easy industrial availability of starting materials, the total number of carbon atoms in R² is preferably from 3 to 10, more preferably from 3 to 8. In R², the alkoxy group part preferably has 1 to 4 carbon atoms, and the alkylene group part preferably has 1 to 8 carbon atoms. Specific examples of the alkoxyalkyl groups include 2-methoxyethyl, 2-ethoxyethyl, 2-propoxyethyl, 2-isopropoxyethyl, 2-butoxyethyl, 2-isobutoxyethyl, 2-tert-butoxyethyl, 1-methoxypropane-2-yl, 1-ethoxypropane-2-yl, 1-propoxypropane-2-yl, 1-isopropoxypropane-2-yl, 1-butoxypropane-2-yl, 1-isobutoxypropane-2-yl, 1-tert-butoxypropane-2-yl, 2-methoxypropyl, 2-ethoxypropyl, 2-propoxypropyl, 2-isopropoxypropyl, 2-butoxypropyl, 2-isobutoxypropyl, 2-tert-butoxypropyl, 5-methoxy-3-oxapentyl, 5-ethoxy-3-oxapentyl, 5-propoxy-3-oxapentyl, 5-isopropoxy-3-oxapentyl, 5-butoxy-3-oxapentyl, 5-isobutoxy-3-oxapentyl, 5-tert-butoxy-3-oxapentyl, 8-methoxy-3,6-dioxaoctyl, 8-ethoxy-3,6-dioxaoctyl, 8-propoxy-3,6-dioxaoctyl, 8-isopropoxy-3,6-dioxaoctyl, 8-butoxy-3,6-dioxaoctyl, 8-isobutoxy-3,6-dioxaoctyl and 8-tert-butoxy-3,6-dioxaoctyl.

More specifically, for the compounds represented by the formula (1), the compounds represented by the following formulas (1a) and (1b) are preferably exemplified:

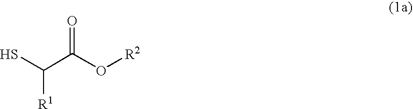

wherein R¹ is a hydrogen atom or an alkyl group of 1 to 5 carbon atoms; and R² is as defined in the formula (1);

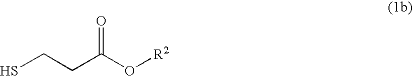

wherein R² is as defined in the formula (1).

In the formula (1a), R¹ is a hydrogen atom or an alkyl group of 1 to 5 carbon atoms, and is preferably a hydrogen atom or an alkyl group of 1 to 4 carbon atoms such as methyl, ethyl, propyl or butyl group. Of these, the hydrogen atom, methyl and ethyl groups are preferable, and the hydrogen atom and methyl group are more preferable in view of easy industrial availability of starting materials.

R² is as defined in the formula (1), with examples including those described in the formula (1). Of these, 2-methoxyethyl, 2-ethoxyethyl, 2-propoxyethyl, 2-methoxy-1-methylethyl, 2-ethoxy-1-methylethyl, 2-methoxypropyl, 2-ethoxypropyl, 5-methoxy-3-oxapentyl and 5-ethoxy-3-oxapentyl are preferred in view of easy industrial availability of starting materials, and 2-methoxyethyl, 2-ethoxyethyl, 2-methoxypropyl, 2-ethoxypropyl, 1-methoxypropane-2-yl, 1-ethoxypropane-2-yl, 5-methoxy-3-oxapentyl and 5-ethoxy-3-oxapentyl are more preferred.

Specific examples of the mercapto compounds represented by the formula (1a) include 2-methoxyethyl thioglycolate, 2-ethoxyethyl thioglycolate, 2-methoxypropyl thioglycolate, 2-ethoxypropyl thioglycolate, 2-methoxyethyl thiolactate, 2-ethoxyethyl thiolactate, 2-methoxypropyl thiolactate and 2-ethoxypropyl thiolactate.

Of these, 2-methoxyethyl thioglycolate, 2-ethoxyethyl thioglycolate, 2-methoxyethyl thiolactate and 2-ethoxyethyl thiolactate are preferable in view of hair relaxing performance and easiness of industrial production.

In the formula (1b), R² is as defined in the formula (1)

Specific examples of the mercapto compounds represented by the formula (1b) include 2-methoxyethyl 3-mercaptopropionate, 2-ethoxyethyl 3-mercaptopropionate, 2-methoxypropyl 3-mercaptopropionate and 2-ethoxypropyl 3-mercaptopropionate.

Of these, 2-methoxyethyl 3-mercaptopropionate and 2-ethoxyethyl 3-mercaptopropionate are preferable in view of hair relaxing performance and easiness of industrial production.

The mercapto compounds of the formula (1), more specifically the alkoxyalkyl mercaptocarboxylates of the formulae (1a) and (1b) can be synthesized from commercially available thiocarboxylates or more easily available thiocarboxylic acids as starting materials by mixing the starting materials with monoalkoxyalkyl alcohols and heating the mixture in the presence of an acid catalyst such as mineral acid or organic acid.

In the formula (2), X is a structure selected from the group consisting of a single bond, —O—, —S—, —NH— and —NR⁴— wherein R⁴ is an alkyl group of 1 to 6 carbon atoms. In view of improving the penetration into hair, R⁴ is preferably a methyl or ethyl group, and is particularly preferably a methyl group in view of easy industrial availability of starting materials. X is preferably —O—, —NH— or —NR⁴—, in which case the solubility in water or aqueous solution is relatively high and the preparation of the relaxer is easy.

Y is an oxygen atom or a sulfur atom, and is preferably an oxygen atom in view of easy industrial availability of starting materials.

R³ is a divalent organic residue having at least one mercapto group (—SH). R³ is not particularly limited as long as it is a divalent organic residue having at least one mercapto group, but is preferably an alkylene group having at least one mercapto group. Preferred examples of the alkylene groups having at least one mercapto group include alkylene groups which have at least one mercapto group and optionally a branch and whose main chain has 2 to 6 carbon atoms. When X is any of —O—, —S—, —NH— and —NR⁴—, R³ is more preferably an ethylene group or a propylene group having at least one mercapto group in view of easy industrial availability of starting materials.

From the viewpoints of easiness of industrial production and handling properties in preparing the hair relaxer, the divalent organic residue preferably has 1 to 3, more preferably 1 to 2 mercapto groups.

There is particularly no limitation on the position of the mercapto groups bonded to the divalent organic residue. The mercapto groups may be bonded to the divalent organic residue directly or through an alkylene group or the like (for example, mercaptoethyl group). When Y is an oxygen atom, the mercapto groups are preferably bonded to the carbon atom at the α-position of the carbonyl group —CY— (this carbon atom is one constituting the divalent organic residue) in view of easy industrial production and hair shaping and relaxing performance.

Specific examples of the mercapto compounds represented by the formula (2) in which X is any of —O—, —S—, —NH— and —NR⁴— include 3-mercapto-4-butanolide, 2,3-dimercapto-4-butanolide, 2,4-dimercapto-4-butanolide, 3,4-dimercapto-4-butanolide, 3-mercapto-4-butyrothiolactone, 3-mercapto-4-butyrolactam, 2,3-dimercapto-4-butyrolactam, 2,4-dimercapto-4-butyrolactam, 3,4-dimercapto-4-butyrolactam, 3-mercapto-5-pentanolide, 4-mercapto-5-pentanolide, 2,3-dimercapto-5-pentanolide, 2,4-dimercapto-5-pentanolide, 2,5-dimercapto-5-pentanolide, 3,4-dimercapto-5-pentanolide, 3-mercapto-5-valerothiolactone, 3-mercapto-5-valerolactam, 4-mercapto-5-valerolactam, 2,3-dimercapto-5-valerolactam, 2,4-dimercapto-5-valerolactam, 2,5-dimercapto-5-valerolactam, 3-mercapto-6-hexanolide, 4-mercapto-6-hexanolide, 5-mercapto-6-hexanolide, 2,3-dimercapto-6-hexanolide, 2,4-dimercapto-6-hexanolide, 2,5-dimercapto-6-hexanolide, 3-mercapto-6-hexanolactam, 4-mercapto-6-hexanolactam, 5-mercapto-6-hexanolactam, 2,3-dimercapto-6-hexanolactam, 2,4-dimercapto-6-hexanolactam, 2,5-dimercapto-6-hexanolactam, 2-mercapto-3-propiolactone, 2-mercapto-2-methyl-3-propiolactone, 2-mercapto-3-methyl-3-propiolactone, 2-mercapto-3-ethyl-3-propiolactone, 2-mercapto-2,3-dimethyl-3-propiolactone, 2-mercapto-3-propiolactam, 2-mercapto-2-methyl-3-propiolactam, 2-mercapto-3-methyl-3-propiolactam, 2-mercapto-3-ethyl-3-propiolactam, 2-mercapto-2,3-dimethyl-3-propiolactam, 2-mercapto-3-propiothiolactone, 2-mercapto-2-methyl-3-propiothiolactone, 2-mercapto-3-methyl-3-propiothiolactone, 2-mercapto-3-ethyl-3-propiothiolactone, 2-mercapto-2,3-dimethyl-3-propiothiolactone, 2-mercapto-4-butanolide, 2-mercapto-2-methyl-4,4-dimethyl-4-butanolide, 2-mercapto-3-(2-propenyl)-4-butanolide, 2-mercapto-4-methyl-4-butanolide, 2-mercapto-2-methyl-4-butanolide, 2-mercapto-3-methyl-4-butanolide, 2-mercapto-4-methyl-4-butanolide, 2-mercapto-3,4-dimethyl-4-butanolide, 2-mercapto-2-ethyl-4-butanolide, 2-mercapto-3-ethyl-4-butanolide, 2-mercapto-4-ethyl-4-butanolide, 2-mercapto-4-butyrothiolactone, 2-mercapto-2-methyl-4-butyrothiolactone, 2-mercapto-3-methyl-4-butyrothiolactone, 2-mercapto-4-methyl-4-butyrothiolactone, 2-mercapto-3,4-dimethyl-4-butyrothiolactone, 2-mercapto-2-ethyl-4-butyrothiolactone, 2-mercapto-3-ethyl-4-butyrothiolactone, 2-mercapto-4-ethyl-4-butyrothiolactone, 2-mercapto-4-butyrolactam, 2-mercapto-2-methyl-4-butyrolactam, 2-mercapto-3-methyl-4-butyrolactam, 2-mercapto-4-methyl-4-butyrolactam, 2-mercapto-3,4-dimethyl-4-butyrolactam, 2-mercapto-2-ethyl-4-butyrolactam, 2-mercapto-3-ethyl-4-butyrolactam, 2-mercapto-4-ethyl-4-butyrolactam, 2-mercapto-5-pentanolide, 2-mercapto-2-methyl-5-pentanolide, 2-mercapto-3-methyl-5-pentanolide, 2-mercapto-4-methyl-5-pentanolide, 2-mercapto-5-methyl-5-pentanolide, 2-mercapto-2-ethyl-5-pentanolide, 2-mercapto-3-ethyl-5-pentanolide, 2-mercapto-4-ethyl-5-pentanolide, 2-mercapto-5-ethyl-5-pentanolide, 2-mercapto-5-valerolactam, 2-mercapto-2-methyl-5-valerolactam, 2-mercapto-3-methyl-5-valerolactam, 2-mercapto-4-methyl-5-valerolactam, 2-mercapto-5-methyl-5-valerolactam, 2-mercapto-2-ethyl-5-valerolactam, 2-mercapto-3-ethyl-5-valerolactam, 2-mercapto-4-ethyl-5-valerolactam, 2-mercapto-5-ethyl-5-valerolactam, 2-mercapto-5-valerothiolactone, 2-mercapto-2-methyl-5-valerothiolactone, 2-mercapto-3-methyl-5-valerothiolactone, 2-mercapto-4-methyl-5-valerothiolactone, 2-mercapto-5-methyl-5-valerothiolactone, 2-mercapto-2-ethyl-5-valerothiolactone, 2-mercapto-3-ethyl-5-valerothiolactone, 2-mercapto-4-ethyl-5-valerothiolactone, 2-mercapto-5-ethyl-5-valerothiolactone, 2-mercapto-6-hexanolide, 2-mercapto-2-methyl-6-hexanolide, 2-mercapto-3-methyl-6-hexanolide, 2-mercapto-4-methyl-6-hexanolide, 2-mercapto-5-methyl-6-hexanolide, 2-mercapto-6-methyl-6-hexanolide, 2-mercapto-6-hexanolactam, 2-mercapto-2-methyl-6-hexanolactam, 2-mercapto-3-methyl-6-hexanolactam, 2-mercapto-4-methyl-6-hexanolactam, 2-mercapto-5-methyl-6-hexanolactam, 2-mercapto-6-methyl-6-hexanolactam, 2-mercapto-6-hexanothiolactone, 2-mercapto-2-methyl-6-hexanothiolactone, 2-mercapto-3-methyl-6-hexanothiolactone, 2-mercapto-4-methyl-6-hexanothiolactone, 2-mercapto-5-methyl-6-hexanothiolactone, 2-mercapto-6-methyl-6-hexanothiolactone and N-methyl or N-ethyl derivatives of these lactams.

Of these, 2-mercapto-4-butanolide, 2-mercapto-4-butyrothiolactone, 2-mercapto-4-butyrolactam, N-methyl-2-mercapto-4-butyrolactam, 2-mercapto-4-methyl-4-butanolide, 2-mercapto-4-ethyl-4-butanolide, 2-mercapto-5-pentanolide, 2-mercapto-5-valerolactam, N-methyl-2-mercapto-5-valerolactam, 2-mercapto-6-hexanolactam, 3-mercapto-4-butanolide, 2,3-dimercapto-4-butanolide, 2,4-dimercapto-4-butanolide, 3-mercapto-4-butyrolactam, 2,3-dimercapto-4-butyrolactam, 2,4-dimercapto-4-butyrolactam, 2,3-dimercapto-5-pentanolide, 2,4-dimercapto-5-pentanolide, 2,5-dimercapto-5-pentanolide, 3-mercapto-5-valerolactam, 4-mercapto-5-valerolactam, 2,3-dimercapto-5-valerolactam, 2,4-dimercapto-5-valerolactam and 2,5-dimercapto-5-valerolactam are preferred, and 2-mercapto-4-butanolide, 2-mercapto-4-methyl-4-butanolide, 2-mercapto-4-ethyl-4-butanolide, 2-mercapto-4-butyrolactam, N-methyl-2-mercapto-4-butyrolactam, 2-mercapto-5-valerolactam, N-methyl-2-mercapto-5-valerolactam and 2-mercapto-6-hexanolactam are particularly preferred in view of hair relaxing performance and easy industrial availability of starting materials.

The mercapto compounds represented by the formula (2) can be produced by known methods. For example, such compounds can be synthesized by halogenating lactone compounds and lactam compounds followed by introduction of mercapto groups.

Specifically, mercaptolactones and mercaptothiolactones may be synthesized by a series of steps in which commercially available lactones or thiolactones are halogenated in accordance with a method described in J. Am. Chem. Soc. 1945, 67. 2218-2220, and the synthesized halides or commercially available halides are produced into objective lactone derivatives by a method described in Ann. 1960, 639. 146-56.

Mercaptolactams may be synthesized by a series of steps in which halides are synthesized by a method described in J. Am. Chem. Soc. 1958. 80. 6233-6237, and the resultant halides are synthesized into objective lactam derivatives by a method described in Ann. 1960, 639. 146-56, similarly to the production of lactones.

In the mercapto compounds represented by the formula (2), it is also preferable that X is a single bond in view of easy availability of starting materials. When X is a single bond, the mercapto compounds are represented by the following formula (2a):

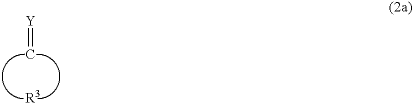

wherein Y and R³ are as defined in the formula (2). When X is a single bond, R³ may be preferably a butylene or a pentylene group having at least one mercapto group. The number of mercapto groups is preferably from 1 to 2 in view of easy handling and production.

Specific examples of the mercapto compounds represented the formula (2a) include 2-mercaptocyclopentanone, 3-mercaptocyclopentanone, 2-mercaptocyclohexanone, 3-mercaptocyclohexanone, 2-mercaptocycloheptanone, 3-mercaptocycloheptanone, 2-mercaptocyclooctanone, 3-mercaptocyclooctanone, 2-mercapto-tetrahydropyran-4-one, 3-mercapto-tetrahydropyran-4-one, 2-mercapto-tetrahydrothiopyran-4-one, 3-mercapto-tetrahydrothiopyran-4-one, 4-mercapto-tetrahydrothiophene-3-one, 5-mercapto-3-pyrrolidone, 5-mercapto-N-methyl-3-pyrrolidone, 4-mercapto-tetrahydropyran-3-one, 5-mercapto-tetrahydropyran-3-one, 3-mercapto-tetrahydropyran-4-one, 4-mercapto-3-piperidone, 5-mercapto-3-piperidone, 3-mercapto-4-piperidone, 4-mercapto-N-methyl-3-piperidone, 5-mercapto-N-methyl-3-piperidone and 3-mercapto-N-methyl-4-piperidone. Of these, 2-mercaptocyclopentanone and 2-mercaptocyclohexanone are preferred.

These mercapto compounds also may be synthesized from commercially available halides by a method described in Ann. 1960, 639. 146-56.

Next, the hair relaxer according to the present invention will be described.

The hair relaxer of the invention contains at least one mercapto compound represented by the formula (1) or (2). The mercapto compounds may be used singly or in combination of two or more kinds.

The hair relaxer generally contains the mercapto compound in an amount of 0.01 to 15% by mass, more preferably 0.1 to 10% by mass, still more preferably 1 to 5% by mass. When the content of the mercapto compound is in this range, the hair relaxer can produce a hair relaxing effect sufficiently as expected.

When the content of the mercapto compound is below the lower limit, the effects of the invention can hardly be obtained. The content exceeding the upper limit results in so strong an odor that the hair relaxer can be unpractical.

The hair relaxer of the invention may be prepared prior to use, or may be prepared on site by mixing agents immediately before use. In the on-site preparation, the mercapto compound of the formula (1) or (2) in undiluted or crystalline form may be added to an agent containing other than the mercapto compound. Alternatively, a solution in which the mercapto compound of the formula (1) or (2) is diluted with an additive such as a swelling agent or a penetration enhancer may be mixed with an agent containing other than the mercapto compound.

When preparing the hair relaxer based on water, common cosmetic additives such as propylene glycol, N-methylpyrrolidone and ethoxy ethanol may be used as solubilizing agents to enhance the solubility in water of the mercapto compounds represented by the formula (1) or (2) and thereby to reduce the dissolution time and prevent the formation of oil phase. Increasing the amount of the solubilizing agent can enhance the dissolution rate. In the event that the increased amount of such additives leads to lowering in the primary performance required, the hair relaxer may be prepared such that it is separated in two phases and is mixed together every time of use. Alternatively, the hair relaxer may be emulsified with use of a surfactant.

The hair relaxer may be favorably used mainly for straightening and uncurling hair and correcting so-called bed hair as well as for creating curls, and does not entail oxidization with bromate or hydrogen peroxide as commonly performed in the field of permanent waving agents.

Applications of the hair relaxer are not particularly limited and include shampoos, rinsing conditioners, conditioners, hair treatments, hair lotions, hair waxes, hair mousses and hair gels.

Of these, the hair relaxer is preferably used as hair cosmetics selected from the group consisting of shampoos, rinsing conditioners, conditioners, hair treatments, hair lotions and hair mousses.

Formulations of the hair relaxer include liquids, foams, gels, creams and pastes. Depending on the formulation, the hair relaxer may be used as various types, including liquid type, spray type, aerosol type, cream type and gel type.

The hair relaxer is capable of relaxing frizzy hair or curled hair in a relatively short time period from when the hair relaxer is applied to hair to when the hair is styled with a comb or washed.

The hair relaxer can be used on the alkaline side needless to say, and can work at pH levels in a weakly acidic to neutral region that are lower than those of the conventional hair processing agents containing sulfites. Moreover, the hair relaxer produces a higher effect of shaping and relaxing hair in that pH region. The pH of the hair relaxer is not particularly limited, and is preferably in the range of 2.5 to 9.0, more preferably 3.5 to 8.0, particularly preferably 4.0 to 7.5. The pH in this range reduces the skin irritations and will not damage hair. The pH is a value measured at 23° C.

Furthermore, the hair relaxer of the present invention improves hair's softness. The reasons for this effect are not clear but are believed to be that the mercapto compound of the formula (1) or (2) increases lipophilicity of the hair relaxer to improve hair penetration properties and consequently the relaxer can produce effects in a short time period, and because the hair relaxer is used in a weakly acidic to neutral region, it does not damage hair and gives hair softness.

Although the hair relaxer can relax hair sufficiently without containing sulfites or bisulfites, it may contain traditional substances such as sulfites, bisulfites, thioglycolic acid and cysteine while still achieving the effects of the invention.

Further, known common additives may be added in order to improve the hair processing performance and the comfort of use of the hair relaxer. Examples of the additives include surfactants, foaming washing assistants, supper fatting agents, thickeners, viscosity modifiers, opacifiers, chelating agents, ultraviolet absorbers, antiseptic agents, anti-scuff agents, sterilizing antiseptic agents, hair protecting agents, wetting agents, emulsifying agents, penetration enhancers, buffers, perfumes, dyes, stabilizers, odor masking agents and pearling agents. Beauty ingredients and common cosmetic ingredients may be added as required.

The surfactants include anionic surfactants such as sodium lauryl sulfate, sodium polyoxyethylene lauryl ether sulfate, methyltaurine sodium coconut fatty acid ester and lauroyl methylalanine sodium; amphoteric surfactants such as betaine lauryldimethylaminoacetate, imidazoline surfactants and amidopropyl betaine coconut fatty acid ester; cationic surfactants such as cetyltrimethylammonium chloride, stearyltrimethylammonium chloride and behenyltrimethylammonium chloride; and nonionic surfactants such as alkyl alkanolamides.

The thickeners include polymer compounds such as carboxymethylcellulose, carboxyvinyl polymers, hydroxyethylcellulose, hydroxypropylcellulose, xanthan gum, carrageenan, alginic acid salts, pectin, tragacanth gum and polyvinylpyrrolidone; higher alcohols such as lauryl alcohol, cetyl alcohol, stearyl alcohol, isostearyl alcohol, oleyl alcohol and behenyl alcohol; kaolin; fatty acids such as lauric acid, myristic acid, palmitic acid, stearic acid, behenic acid, oleic acid, undecylic acid and isostearic acid; and vaseline.

The wetting agents and emulsifying agents include glycerin, diglycerin, propylene glycol, dipropylene glycol, 1,3-butanediol, polyethylene glycol, sorbitol, plant extracts, vitamins, hyaluronic acid salts, chondroitin sulfate, the above-described cationic, anionic, amphoteric and nonionic surfactants, ether nonionic surfactants such as polyoxyethylene oleyl ether, polyoxyethylene stearyl ether, polyoxyethylene cetyl ether, polyoxyethylene octylphenyl ether, polyoxyethylene dodecylphenyl ether and polyoxyethylene nonyl ether, dimethylpolysiloxane, methylphenylpolysiloxane, and silicone derivatives such as amino-modified silicone oils, alcohol-modified silicone oils, fluorine-modified silicone oils, polyether-modified silicone oils and alkyl-modified silicone oils.

The penetration enhancers include ethanol, propanol, isopropanol, 1,2-propylene glycol, 1,3-butanediol, glycerin, ethylcarbitol, benzyl alcohol, benzyloxyethanol, urea and 2-methylpyrrolidone.

The buffers include inorganic buffers, buffers containing basic amino acids such as arginine and lysine, and organic acids such as citrates.

The pH adjusters include inorganic acids such as hydrochloric acid and phosphoric acid; inorganic acid salts such as disodium hydrogen phosphate and sodium dihydrogen phosphate; organic acids such as citric acid, malic acid, lactic acid, succinic acid and oxalic acid, and sodium salts of the acids; and alkaline agents such as ammonia, diethanolamine, triethanolamine, sodium hydroxide, potassium hydroxide, sodium carbonate, sodium hydrogen carbonate, potassium carbonate and potassium hydrogen carbonate.

For the perfumes, (A) hydrocarbons, (B) alcohols, (C) phenols, (D) aldehydes and/or acetals, (E) ketones and/or ketals, (F) ethers, (G) synthetic musks, (H) acids, (I) lactones, (J) esters, (K) nitrogen-containing and/or sulfur-containing and/or halogen-containing compounds, and (L) natural perfumes can be used. Specific examples of the perfumes include those disclosed in JP-A-2003-137758.

The hydrocarbons (A) are not particularly limited as long as they are volatile organic compounds composed of carbon and hydrogen. Examples thereof include aliphatic hydrocarbons, alicyclic hydrocarbons, terpene hydrocarbons and aromatic hydrocarbons.

The alcohols (B) are not particularly limited as long as they are volatile organic compounds with hydroxyl groups. Examples thereof include aliphatic alcohols, alicyclic alcohols, terpene alcohols and aromatic alcohols.

The phenols (C) are not particularly limited as long as they are organic phenolic compounds or derivatives thereof having aroma. Examples thereof include monovalent, divalent and trivalent phenolic compounds, polyphenols, and ether derivatives of these compounds.

The aldehydes and acetals (D) are not particularly limited as long as they are volatile organic compounds having aldehyde or acetal groups in the molecule. Examples thereof include aliphatic aldehydes and acetals, terpene aldehydes and acetals, and aromatic aldehydes and acetals.

The ketones and ketals (E) are not particularly limited as long as they are volatile organic compounds having ketone or ketal groups in the molecule. Examples thereof include aliphatic ketones and ketals, terpene ketones and ketals, and aromatic ketones and ketals.

The ethers (F) are not particularly limited as long as they are volatile organic compounds having ether groups in the molecule. Examples thereof include aliphatic ethers, terpene ethers and aromatic ethers.

The synthetic musks (G) are not particularly limited as long as they are organic compounds having musk scent or similar scent.

The acids (H) are not particularly limited as long as they are organic compounds having carboxyl groups in the molecule.

The lactones (I) are not particularly limited as long as they are volatile organic compounds having lactone groups in the molecule.

The esters (J) are not particularly limited as long as they are volatile organic compounds having ester groups in the molecule.

The nitrogen-containing and/or sulfur-containing and/or halogen-containing compounds (K) are not particularly limited as long as they are fragrant organic compounds containing nitrogen, sulfur and halogen in the molecule.

The natural perfumes (L) are not particularly limited.

Specific examples of these perfumes include 2,6,10-trimethyl-9-undecanal, n-decenal, n-octanal, allyl amyl glycolate, allyl hexanoate, α-amyl cinnamyl aldehyde, anethole, p-methoxybenzaldehyde, 6,7-dehydro-1,1,2,3,3-pentamethyl-4(5H)-indanone, cinnamyl alcohol, cis-3-hexene-1-yl acetate, cis-6-nonenol, citral, citral diethyl acetal, citronellal, citronellol, citronellyl nitrile, tricyclodecene acetate, tricyclodecene propionate, cyclohexyloxy-2-propenyl acetate, δ-damascone, dihydrojasmone, diphenyl ether, acetaldehyde ethyl phenyl acetal, acetaldehyde ethyl linalyl acetal, estragole, ethyl 2-methylbutyrate, ethyl maltol, ethyl butyrate, ethyl dehydrocyclogeranate, ethylvanillin, eugenol, p-ethyl-α,α-dimethyldihydrocinnamaldehyde, 2,4,6-trimethyl-4-phenyl-1,3-dioxane, ethyloctahydro-4,7-methano[3aH]-3a-carboxylate, 2-ethyl-4-hydroxy-5-methyl-3-(2H)furanone, α-methylphenylacetaldehyde, indole, α-ionone, isoamyl salicylate, isobutylquinoline, α-isomethylionone, phenylacetaldehyde, 6-(3-phenyl)tetrahydro[2H]pyran-2-one, 2-(2,4-dimethyl-3-cyclohexyl)-5-methyl-5-(1-methylpropyl)-1,3-dioxane, cis-3-hexenol, geranyl nitrile, cis-3-hexenyl methyl carbonate, tetrahydrolinalool, linalool, cis-3-dodecenal, ethyl 2-methylpentanoate, 2,6-dimethyl-5-heptanal, methyl anthranilate, methyl benzoate, methyl salicylate, 3-methylcyclopentadecanolide, nerol, p-cresol, p-methylanisole, phenylacetic acid, β-phenethyl alcohol, phenethyl formate, phenethyl isoamyl ether, 4-(p-hydroxyphenyl)-2-butanone, tetrahydro-4-methyl-2-(2-methyl-1-propenyl)-(2H)pyran, 5-methyl-3-heptanone oxime, trans-2-hexene-1-ol, 2-tridecenonitrile, 3-cyclohexene-1-carboxaldehyde dimethyl acetal, vanillin, o-tert-butylcyclohexyl acetate, acetyl cedrene, vetiveryl acetate, phenylacetaldehyde dimethyl acetal and rodinol.

Other additives include lubricants such as paraffin, liquid paraffin, beeswax, squalane, jojoba oil, olive oil, ester oil, triglyceride, vaseline and lanoline; and hair protecting agents such as collagen and keratin hydrolysates and derivatives thereof.

### EXAMPLES

The present invention will be described with reference to the following examples, but it should be construed that the invention is in no way limited to the examples. In Examples below, all percentages and parts are by mass unless otherwise mentioned.

### Synthetic Example 1

### Synthesis of 2-methoxyethyl thioglycolate

A 1000-ml four-necked flask equipped with a thermometer and a condenser tube was charged with 300 g of methyl thioglycolate (2.83 mol, manufactured by Tokyo Kasei Kogyo Co., Ltd.), 320 g of 2-methoxyethanol (4.21 mol, manufactured by Tokyo Kasei Kogyo Co., Ltd.) and 3.6 g of 95% sulfuric acid (manufactured by JUNSEI CHEMICAL CO., LTD.), followed by stirring at 80° C. for 5 hours. During the reaction, the pressure was slightly reduced by means of an aspirator connected at an upper part of the condenser tube, and methanol formed as the reaction proceeded was distilled away. After the reaction, the liquid was directly concentrated and purified by distillation under reduced pressure to give 123 g (0.82 mol, 29% yield) of 2-methoxyethyl thioglycolate at a boiling point of 65° C. (0.6 kPa).

### Synthetic Example 2

### Synthesis of 2-ethoxyethyl thioglycolate

The procedures in Synthetic Example 1 were repeated except that 300 g of methyl thioglycolate (2.83 mol, manufactured by Tokyo Kasei Kogyo Co., Ltd.) and 379 g of 2-ethoxyethanol (4.21 mol, manufactured by Tokyo Kasei Kogyo Co., Ltd.) were used. Consequently, 144 g (0.88 mol, 31% yield) of 2-ethoxyethyl thioglycolate was obtained at a boiling point of 99-103° C. (2.0 kPa).

### Synthetic Example 3

### Synthesis of 2-ethoxyethyl thiolactate

The procedures in Synthetic Example 1 were repeated except that 300 g of thiolactic acid (2.83 mol) and 379 g of 2-ethoxyethanol (4.21 mol, manufactured by Tokyo Kasei Kogyo Co., Ltd.) were used. Consequently, 175 g (0.98 mol, 35% yield) of 2-ethoxyethyl thiolactate was obtained at a boiling point of 102° C. (2.6 kPa).

### Synthetic Example 4

### Synthesis of 2-ethoxyethyl mercaptopropionate

The procedures in Synthetic Example 1 were repeated except that 300 g of mercaptopropionic acid (2.83 mol) and 379 g of 2-ethoxyethanol (4.21 mol, manufactured by Tokyo Kasei Kogyo Co., Ltd.) were used. Consequently, 166 g (0.93 mol, 33% yield) of 2-ethoxyethyl mercaptopropionate was obtained at a boiling point of 101° C. (2.0 kPa).

### Synthetic Example 5

### Synthesis of 2-mercapto-4-butanolide (other name: 2-mercapto-4-butyrolactone)

70% Sodium hydrosulfide (49 g, 0.6 mmol, manufactured by JUNSEI CHEMICAL CO., LTD.) was dissolved in methyl alcohol (500 g, special grade, manufactured by JUNSEI CHEMICAL CO., LTD.) and purified water (500 g, water distilled and passed through an ion exchange filter). The resultant solution was cooled with ice to not more than 10° C. with stirring. To the cooled solution, 2-bromo-4-butanolide (100 g, 0.6 mol, manufactured by Tokyo Kasei Kogyo Co., Ltd.) was added dropwise over a period of about 30 minutes. After the completion of the dropwise addition, the liquid was stirred for 10 minutes, and the resultant reaction liquid was concentrated to approximately half of the original volume under reduced pressure. To the concentrated liquid was added ethyl acetate (500 ml, special grade, manufactured by JUNSEI CHEMICAL CO., LTD.) followed by extraction. The aqueous phase obtained was subjected to re-extraction with ethyl acetate (500 ml). The organic phases thus extracted were combined and concentrated and purified by distillation under reduced pressure to give 2-mercapto-4-butanolide (23 g, 32% yield) at a boiling point of 94° C. (0.3 kPa).

### Synthetic Example 6

### Synthesis of 2-mercaptocyclopentanone

10 Grams of hydrogen sulfide gas (bottled hydrogen sulfide gas manufactured by SUMITOMO SEIKA CHEMICALS CO., LTD.) was blown into 44 g of a methanol solution of sodium methoxide (28% concentration and 0.22 mol in terms of sodium methoxide, manufactured by JUNSEI CHEMICAL CO., LTD.) while keeping the temperature at approximately not more than 10° C. by cooling with ice. While still keeping the temperature at not more than 10° C., 23.7 g of 2-bromocyclopentanone (0.2 mol, manufactured by Tokyo Kasei Kogyo Co., Ltd.) was added dropwise. After the completion of the dropwise addition, the liquid was stirred for 30 minutes while keeping the temperature at not more than 5° C. After the reaction, the system was gradually evacuated with a vacuum pump, and methanol was distilled away until the liquid weighed approximately 35 g. The residual liquid after distillation was adjusted to pH 3 by dropwise addition of 10% hydrochloric acid while keeping the temperature at not more than 10° C. After the pH adjustment, the liquid was subjected to extraction by adding thereto 100 g of diethyl ether. The aqueous phase obtained was subjected to re-extraction two times, each with 100 g of diethyl ether. The diethyl ether phases extracted were mixed together and concentrated using an evaporator. The concentrated oily substance was purified by distillation under reduced pressure to give 7.4 g (0.064 mol, 32% yield) of 2-mercaptocyclopentanone at a boiling point of 51° C. (0.9 kPa).

### Synthetic Example 7

### Synthesis of 2,4-dibromobutanoyl bromide

2,4-Dibromobutanoyl bromide was synthesized from 4-butanolide according to a method of A. Kamal, et al. (Tetrahedron: Asymmetry 2003, 14, 2587).

Specifically, phosphorus tribromide (2.5 g, 0.43 g atom, manufactured by Tokyo Kasei Kogyo Co., Ltd.) was added to 4-butanolide (20 g, 0.23 mol, manufactured by Tokyo Kasei Kogyo Co., Ltd.).

To the resultant solution, bromine (40.4 g, 0.25 mol, manufactured by Wako Pure Chemical Industries, Ltd.) was added dropwise with stirring over a period of about 2 hours while keeping the temperature at not more than 10° C. After the completion of the dropwise addition, the mixture was heated to 70° C., and bromine (40.4 g, 0.25 mol, manufactured by Wako Pure Chemical Industries, Ltd.) was added dropwise over a period of about 30 minutes. After the completion of the dropwise addition, the liquid was heated to 80° C. and stirred at 80° C. for 3 hours.

After the completion of the reaction, a glass tube was inserted to the bottom of the reaction liquid, and nitrogen was blown into the liquid through the glass tube to remove unreacted bromine and hydrogen bromide formed by the reaction. The reaction liquid was then distilled under reduced pressure to give 2,4-dibromobutanoyl bromide (38 g, 0.12 mol, bp: 87-88° C./0.7 kPa, 53% yield).

### Synthetic Example 8

### Synthesis of N-methyl-2-bromo-4-butyrolactam

A solution mixture consisting of 40% aqueous methylamine solution (7.9 g, 0.10 mol, manufactured by JUNSEI CHEMICAL CO., LTD.) and water (3.3 g) was cooled to not more than 10° C. To the solution mixture, 2,4-dibromobutanoyl bromide (38 g, 0.12 mol) was added dropwise over a period of 15 minutes while keeping the temperature at not more than 10° C. After the completion of the dropwise addition, the mixture was heated to 30° C. and stirred for 30 minutes. The reaction liquid was poured into 50 g of chloroform, and the organic phase was extracted. The organic phase was separated and combined with magnesium sulfate to be dried. Then the magnesium sulfate was filtered out. The organic phase obtained was concentrated, and the concentrate was purified by silica gel column chromatography to give N-methyl-2,4-dibromobutanamide (23.1 g, 0.090 mol, 74% yield).

N-methyl-2,4-dibromobutanamide obtained above was dissolved in THF (200 ml), and the solution was cooled with ice to not more than 10° C. To the cooled solution, 60% NaH in mineral oil (6.6 g, 0.166 mol, manufactured by JUNSEI CHEMICAL CO., LTD.) was added little by little over a period of about 15 minutes. After the completion of the addition, the mixture was heated to room temperature and stirred for 2 hours. After the reaction, the resultant reaction liquid was concentrated to about ⅓ of the original weight, and the concentrate was poured into ice water (100 g). Subsequently, extraction was performed with 100 g of chloroform, and the chloroform phase was concentrated. The concentrate was purified by silica gel column chromatography to give N-methyl-2-bromo-4-butyrolactam (10.2 g, 0.057 mol, 69% yield).

### Synthetic Example 9

### Synthesis of N-methyl-2-mercapto-4-butyrolactam

70% Sodium hydrosulfide (6.1 g, 0.077 mmol, manufactured by JUNSEI CHEMICAL CO., LTD.) was dissolved in methyl alcohol (100 g, special grade, manufactured by JUNSEI CHEMICAL CO., LTD.) and purified water (100 g, water distilled and passed through an ion exchange filter). The resultant solution was cooled with ice to not more than 10° C. with stirring. To the cooled solution, a liquid mixture consisting of N-methyl-2-bromo-4-butyrolactam (11.4 g, 0.064 mol, 77% yield) and methyl alcohol (50 g) was added dropwise over a period of about 30 minutes. After the completion of the dropwise addition, the liquid was stirred for 60 minutes, and the resultant reaction liquid was concentrated to approximately ⅓ of the original volume under reduced pressure. To the concentrated liquid was added ethyl acetate (500 ml; special grade, manufactured by JUNSEI CHEMICAL CO., LTD.) followed by extraction. The aqueous phase obtained was subjected to re-extraction with ethyl acetate (500 ml). The organic phases (ethyl acetate phases) thus extracted were combined and concentrated under reduced pressure. The concentrate was purified by silica gel column chromatography to give N-methyl-2-mercapto-4-butyrolactam (5.4 g, 0.041 mol, 64% yield).

### Synthetic Example 10

### Synthesis of 2-bromo-4-butyrolactam

2,4-Dibromobutanamide (12.4 g, 0.076 mol, mp: 79° C., 63% yield) was produced according to Synthetic Example 8, except that 2,4-dibromobutanoyl bromide obtained as described in Synthetic Example 7 was used and the 40% aqueous methylamine solution was replaced with ammonia water. Subsequently, 2-bromo-4-butyrolactam (3.4 g, 0.021 mol, 27% yield) was produced according to Synthetic Example 8, except that N-methyl-2,4-dibromobutanamide was replaced with above-produced 2,4-dibromobutanamide.

### Synthetic Example 11

### Synthesis of 2-mercapto-4-butyrolactam

2-Mercapto-4-butyrolactam (1.7 g, 0.014 mol, 69% yield) was produced according to Synthetic Example 9, except that N-methyl-2-bromo-4-butyrolactam was replaced with 2-bromo-4-butyrolactam (3.4 g, 0.021 mol).

### [Frizzy Hair Sample]

Naturally (chemically untreated) frizzy hair of twenties age Japanese women was bundled by ten strands by binding their roots to fabricate hair bundles 20 cm long as frizzy hair samples.

The hair bundles were used for evaluating shampoos, rinsing conditioners and hair lotions.

### Examples 1 to 3

### Preparation of Shampoos

Shampoos were prepared by the procedures described below according to the compositions shown in Table 1.

25 Grams of purified water was heated to 70° C., to which an aqueous solution of lauryl polyoxyethylene sulfate triethanolamine salt, an aqueous solution of lauryl polyoxyethylene sulfate sodium salt, lauroyl diethanolamide and polyethylene glycol 400 were added in the order named with stirring. When the mixture became uniform, it was cooled naturally and the pH was adjusted by addition of citric acid and disodium hydrogen phosphate with stirring. 2-Methoxyethyl thioglycolate obtained in Synthetic Example 1 was added to the pH-adjusted liquid, and the mixture was stirred sufficiently. Thereafter, the pH was readjusted, and purified water was added so that the amount of the pH-adjusted shampoo became 100 g, followed by stirring with a glass rod to uniformity.

### [Measurement of Frizziness Before Treatment]

The frizzy hair sample was soaked in a 0.5% aqueous solution of sodium lauryl sulfate (EMAL 2F paste, manufactured by KAO CORPORATION) at 40° C. for 30 minutes, and rinsed twice in approximately 25° C. water. The hair bundle was then lightly towel dried and air-dried at approximately 25° C. in a suspended state with its bound end upside.

Each of the ten strands of dried frizzy hair was measured for length from the upper to the lower end in a suspended state (L1 cm). Subsequently, the hair strands were each pulled straight and measured for length (L0 cm).

The frizziness before treatment was calculated by the following formula:

Frizziness before treatment=L1 cm/L0 cm

The more the frizziness approaches 1 (one), the straighter the hair.

### [Frizz Relaxing]

The frizzy hair sample was placed on a glass plate, and 1 g of the shampoo was dropped on the hair at approximately 1 cm intervals with use of a Pasteur pipette. The shampoo droplets were evenly spread over the hair with a glass rod to wet the hair sufficiently, and the hair was combed straight. The combed hair was then covered with a polyvinylidene chloride wrapping film (product name: Saran Wrap™, manufactured by Asahi Kasei Corporation). The treated hair on the glass plate was allowed to stand in a constant temperature oven at 35° C. for 20 minutes. Thereafter, the hair bundle was removed from the glass plate and was rinsed twice in approximately 25° C. water. The hair bundle was lightly towel dried and air-dried at approximately 25° C. in a suspended state with its bound end upside.

Each of the ten strands of dried frizzy hair was measured for length from the upper to the lower end in a suspended state (L3 cm). Subsequently, the hair strands were each pulled straight and measured for length (L2 cm).

The frizziness after treatment was calculated by the following formula:

Frizziness after treatment=L3 cm/L2 cm

The improvement rate of frizziness was calculated by the following formula, in which the frizziness values were the averages of the ten strands before and after the treatment.

Frizziness improvement rate (%)=[(frizziness after treatment−frizziness before treatment)÷frizziness before treatment]×100

### [Hair Break Test]

### [Test of Hair Break Before Treatment]

A commercially available tension gauge (round (bar) gauge O-BT, manufactured by OBA KEIKI SEISAKUSHO CO., LTD.) was immovably fixed horizontally to the table surface. One strand of hair was hanged in a U-shape on an L-shaped metallic part of the tension gauge. Both ends of the hair were held by hand and were slowly pulled down until the hair was broken. At breakage, the tension gauge stopped and the value was recorded.

Fifty strands of untreated hair were tested in a similar manner and the average was obtained as hair strength before treatment (W0).

### [Test of Hair Break After Treatment]

The hair was treated with the shampoo repeatedly ten times to afford samples for hair break testing. Ten strands of the treated dry frizzy hair were tested using the tension gauge in a manner similar to that used in the hair break test before treatment. The measured values were averaged to determine the hair strength after treatment (W1).

The lowering rate of breaking strength was calculated by the following formula.

Lowering rate of breaking strength (%)=[(W0−W1)÷W0]×100

The results are shown in Table 1.

### Examples 4 to 6

The procedures in Examples 1 to 3 were repeated except that the mercapto compound was replaced with 2-ethoxyethyl thioglycolate obtained in Synthetic Example 2 and the composition was altered as shown in Table 1. The results are shown in Table 1.

### Examples 7 to 9

The procedures in Examples 1 to 3 were repeated except that the mercapto compound was replaced with 2-ethoxyethyl thiolactate obtained in Synthetic Example 3 and the composition was altered as shown in Table 1. The results are shown in Table 1.

### Examples 10 to 12

The procedures in Examples 1 to 3 were repeated except that the mercapto compound was replaced with 2-ethoxyethyl 3-mercaptopropionate obtained in Synthetic Example 4 and the composition was altered as shown in Table 2. The results are shown in Table 2.

### Examples 13 to 15

The procedures in Examples 1 to 3 were repeated except that the mercapto compound was replaced with 2-mercapto-4-butanolide obtained in Synthetic Example 5 and the composition was altered as shown in Table 2. The results are shown in Table 2.

### Examples 16 to 18

The procedures in Examples 1 to 3 were repeated except that the mercapto compound was replaced with 2-mercaptocyclopentanone obtained in Synthetic Example 6 and the composition was altered as shown in Table 2. The results are shown in Table 2.

### Examples 19 to 21

The procedures in Examples 1 to 3 were repeated except that the mercapto compound was replaced with N-methyl-2-mercapto-4-butyrolactam obtained in Synthetic Example 9 and the composition was altered as shown in Table 3. The results are shown in Table 3.

### Examples 22 to 24

The procedures in Examples 1 to 3 were repeated except that the mercapto compound was replaced with 2-mercapto-4-butyrolactam obtained in Synthetic Example 11 and the composition was altered as shown in Table 3. The results are shown in Table 3.

### Comparative Examples 1 to 3

The procedures in Examples 1 to 3 were repeated except that the mercapto compound was replaced with sodium sulfite (manufactured by JUNSEI CHEMICAL CO., LTD.) and the composition was altered as shown in Table 3. The results are shown in Table 3.

TABLE 1
Shampoo (1)

| Ingredients | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| 2-Methoxyethyl thioglycolate | 3.8% | 3.8% | 3.8% | — | — | — | — | — | — |
| 2-Ethoxyethyl thioglycolate | — | — | — | 4.2% | 4.2% | 4.2% | — | — | — |
| 2-Ethoxyethyl thiolactate | — | — | — | — | — | — | 4.5% | 4.5% | 4.5% |
| 2-Ethoxyethyl 3-mercaptopropionate | — | — | — | — | — | — | — | — | — |
| 2-Mercapto-4-butanolide | — | — | — | — | — | — | — | — | — |
| 2-Mercaptocyclopentanone | — | — | — | — | — | — | — | — | — |
| N-methyl-2-mercapto-4-butyrolactam | — | — | — | — | — | — | — | — | — |
| 2-Mercapto-4-butyrolactam | — | — | — | — | — | — | — | — | — |
| Sodium sulfite | — | — | — | — | — | — | — | — | — |
| Lauryl polyoxyethylene (3) sulfate | 32% | 32% | 32% | 32% | 32% | 32% | 32% | 32% | 32% |
| triethanolamine salt (27% aqueous | | | | | | | | | |
| solution) *1 | | | | | | | | | |
| Lauryl polyoxyethylene (3) sulfate sodium | 23% | 23% | 23% | 23% | 23% | 23% | 23% | 23% | 23% |
| salt (27% aqueous solution) *2 | | | | | | | | | |
| Lauroyl diethanolamide *3 | 4% | 4% | 4% | 4% | 4% | 4% | 4% | 4% | 4% |
| Polyethylene glycol 400 *4 | 1% | 1% | 1% | 1% | 1% | 1% | 1% | 1% | 1% |
| Purified water | Balance | Balance | Balance | Balance | Balance | Balance | Balance | Balance | Balance |
| Total | 100% | 100% | 100% | 100% | 100% | 100% | 100% | 100% | 100% |
| pH | 4.1 | 7.0 | 8.9 | 4.0 | 7.1 | 9.0 | 4.1 | 7.0 | 9.0 |
| Frizziness improvement rate | 18% | 18% | 16% | 18% | 17% | 14% | 16% | 15% | 13% |
| Lowering rate of breaking strength | 6% | 14% | 27% | 3% | 12% | 24% | 3% | 10% | 21% |

*1: KAO CORPORATION EMAL 20T
*2: KAO CORPORATION EMAL E-27C
*3: KAO CORPORATION AMINON L-02
*4: KANTO CHEMICAL CO., INC.

TABLE 2
Shampoo (2)

| Ingredients | Ex. 10 | Ex. 11 | Ex. 12 | Ex. 13 | Ex. 14 | Ex. 15 | Ex. 16 | Ex. 17 | Ex. 18 |
|---|---|---|---|---|---|---|---|---|---|
| 2-Methoxyethyl thioglycolate | — | — | — | — | — | — | — | — | — |
| 2-Ethoxyethyl thioglycolate | — | — | — | — | — | — | — | — | — |
| 2-Ethoxyethyl thiolactate | — | — | — | — | — | — | — | — | — |
| 2-Ethoxyethyl 3-mercaptopropionate | 4.9% | 4.9% | 4.9% | — | — | — | — | — | — |
| 2-Mercapto-4-butanolide | — | — | — | 3.0% | 3.0% | 3.0% | — | — | — |
| 2-Mercaptocyclopentanone | — | — | — | — | — | — | 2.9% | 2.9% | 2.9% |
| N-methyl-2-mercapto-4-butyrolactam | — | — | — | — | — | — | — | — | — |
| 2-Mercapto-4-butyrolactam | — | — | — | — | — | — | — | — | — |
| Sodium sulfite | — | — | — | — | — | — | — | — | — |
| Lauryl polyoxyethylene (3) sulfate | 32% | 32% | 32% | 32% | 32% | 32% | 32% | 32% | 32% |
| triethanolamine salt (27% aqueous | | | | | | | | | |
| solution) *1 | | | | | | | | | |
| Lauryl polyoxyethylene (3) sulfate sodium | 23% | 23% | 23% | 23% | 23% | 23% | 23% | 23% | 23% |
| salt (27% aqueous solution) *2 | | | | | | | | | |
| Lauroyl diethanolamide *3 | 4% | 4% | 4% | 4% | 4% | 4% | 4% | 4% | 4% |
| Polyethylene glycol 400 *4 | 1% | 1% | 1% | 1% | 1% | 1% | 1% | 1% | 1% |
| Purified water | Balance | Balance | Balance | Balance | Balance | Balance | Balance | Balance | Balance |
| Total | 100% | 100% | 100% | 100% | 100% | 100% | 100% | 100% | 100% |
| pH | 4.0 | 6.9 | 8.9 | 4.1 | 7.0 | 9.0 | 4.0 | 7.0 | 9.0 |
| Frizziness improvement rate | 16% | 16% | 14% | 16% | 15% | 13% | 16% | 16% | 15% |
| Lowering rate of breaking strength | 9% | 14% | 22% | 5% | 12% | 25% | 5% | 10% | 23% |

*1: KAO CORPORATION EMAL 20T
*2: KAO CORPORATION EMAL E-27C
*3: KAO CORPORATION AMINON L-02
*4: KANTO CHEMICAL CO., INC.

TABLE 3
Shampoo (3)

| Ingredients | Ex. 19 | Ex. 20 | Ex. 21 | Ex. 22 | Ex. 23 | Ex. 24 | Comp. Ex. 1 | Comp. Ex. 2 | Comp. Ex. 3 |
|---|---|---|---|---|---|---|---|---|---|
| 2-Methoxyethyl thioglycolate | — | — | — | — | — | — | — | — | — |
| 2-Ethoxyethyl thioglycolate | — | — | — | — | — | — | — | — | — |
| 2-Ethoxyethyl thiolactate | — | — | — | — | — | — | — | — | — |
| 2-Ethoxyethyl 3-mercaptopropionate | — | — | — | — | — | — | — | — | — |
| 2-Mercapto-4-butanolide | — | — | — | — | — | — | — | — | — |
| 2-Mercaptocyclopentanone | — | — | — | — | — | — | — | — | — |
| N-methyl-2-mercapto-4-butyrolactam | 3.2% | 3.2% | 3.2% | — | — | — | — | — | — |
| 2-Mercapto-4-butyrolactam | — | — | — | 3.0% | 3.0% | 3.0% | — | — | — |
| Sodium sulfite | — | — | — | — | — | — | 3.2% | 3.2% | 3.2% |
| Lauryl polyoxyethylene (3) sulfate | 32% | 32% | 32% | 32% | 32% | 32% | 32% | 32% | 32% |
| triethanolamine salt (27% aqueous | | | | | | | | | |
| solution) *1 | | | | | | | | | |
| Lauryl polyoxyethylene (3) sulfate sodium | 23% | 23% | 23% | 23% | 23% | 23% | 23% | 23% | 23% |
| salt (27% aqueous solution) *2 | | | | | | | | | |
| Lauroyl diethanolamide *3 | 4% | 4% | 4% | 4% | 4% | 4% | 4% | 4% | 4% |
| Polyethylene glycol 400 *4 | 1% | 1% | 1% | 1% | 1% | 1% | 1% | 1% | 1% |
| Purified water | Balance | Balance | Balance | Balance | Balance | Balance | Balance | Balance | Balance |
| Total | 100% | 100% | 100% | 100% | 100% | 100% | 100% | 100% | 100% |
| pH | 4.0 | 7.0 | 8.9 | 4.2 | 6.9 | 9.0 | 4.1 | 7.1 | 9.0 |
| Frizziness improvement rate | 13% | 15% | 18% | 14% | 16% | 17% | 2% | 8% | 13% |
| Lowering rate of breaking strength | 7% | 9% | 11% | 8% | 10% | 12% | 5% | 8% | 16% |

*1: KAO CORPORATION EMAL 20T
*2: KAO CORPORATION EMAL E-27C
*3: KAO CORPORATION AMINON L-02
*4: KANTO CHEMICAL CO., INC.

### Example 25

### Preparation of Rinsing Conditioners

Rinsing conditioners were prepared by the procedures described below according to the compositions shown in Table 4.

Glycerin was added to 60 g of purified water and the mixture was heated to 70° C. The mixture was kept at 70° C. to give an aqueous phase. In a separate vessel, cetyl alcohol, silicone oil, polyoxyethylene oleyl ether and stearyltrimethylammonium chloride were mixed together and liquefied by heating at 70° C. (oil phase). The aqueous phase was added to the oil phase with vigorous stirring.

The mixture was further stirred with cooling, and the pH was adjusted by addition of citric acid and disodium hydrogen phosphate with stirring. 2-Ethoxyethyl thioglycolate obtained in Synthetic Example 2 was added to the pH-adjusted liquid, and the mixture was stirred sufficiently. Thereafter, the pH was readjusted, and purified water was added so that the amount of the pH-adjusted rinsing conditioner became 100 g, followed by stirring with a glass rod to uniformity.

### [Measurement of Frizz Relaxing Effect]

The frizzy hair sample was placed on a glass plate, and 1 g of the rinsing conditioner was dropped on the hair at approximately 1 cm intervals with use of a Pasteur pipette. The rinsing conditioner droplets were evenly spread over the hair with a glass rod to wet the hair sufficiently, and the hair was combed straight.

The combed hair was then covered with a polyvinylidene chloride wrapping film (product name: Saran Wrap™, manufactured by Asahi Kasei Corporation). The treated hair on the glass plate was allowed to stand in a constant temperature oven at 35° C. for 20 minutes. Thereafter, the hair bundle was removed from the glass plate and was rinsed once in approximately 25° C. water. The hair bundle was lightly towel dried and air-dried at approximately 25° C. in a suspended state with its bound end upside.

Thereafter, the dry frizzy hair was measured for posttreatment frizziness in a manner similar to that used for the shampoo treatment evaluation. Separately, the hair was treated with the rinsing conditioner repeatedly ten times, and the hair break was tested in a manner similar to that used for the shampoo treatment evaluation. The results are shown in Table 4.

### Example 26

The procedures in Example 25 were repeated except that the mercapto compound was replaced with 2-ethoxyethyl thiolactate obtained in Synthetic Example 3 and the composition was altered as shown in Table 4. The results are shown in Table 4.

### Example 27

The procedures in Example 25 were repeated except that the mercapto compound was replaced with 2-mercapto-4-butanolide obtained in Synthetic Example 5 and the composition was altered as shown in Table 4. The results are shown in Table 4.

### Example 28

The procedures in Example 25 were repeated except that the mercapto compound was replaced with 2-mercaptocyclopentanone obtained in Synthetic Example 6 and the composition was altered as shown in Table 4. The results are shown in Table 4.

### Example 29

The procedures in Example 25 were repeated except that the mercapto compound was replaced with 2-mercapto-4-butyrolactam obtained in Synthetic Example 11 and the composition was altered as shown in Table 4. The results are shown in Table 4.

### Comparative Examples 4 and 5

The procedures in Example 25 were repeated except that the mercapto compound was replaced with sodium sulfite and the composition was altered as shown in Table 4. The results are shown in Table 4.

TABLE 4
Rinsing conditioner

| Ingredients | Ex. 25 | Ex. 26 | Ex. 27 | Ex. 28 | Ex. 29 | Comp. Ex. 4 | Comp. Ex. 5 |
|---|---|---|---|---|---|---|---|
| 2-Ethoxyethyl thioglycolate | 3.8% | — | — | — | — | — | — |
| 2-Ethoxyethyl thiolactate | — | 4.5% | — | — | — | — | — |
| 2-Mercapto-4-butanolide | — | — | 3.0% | — | — | — | — |
| 2-Mercaptocyclopentanone | — | — | — | 2.9% | — | — | — |
| 2-Mercapto-4-butyrolactam | — | — | — | — | 3.2% | — | — |
| Sodium sulfite | — | — | — | — | — | 3.2% | 3.2% |
| Stearyltrimethylammonium chloride *1 | 2% | 2% | 2% | 2% | 2% | 2% | 2% |
| Cetyl alcohol *2 | 2% | 2% | 2% | 2% | 2% | 2% | 2% |
| Silicon oil *3 | 3% | 3% | 3% | 3% | 3% | 3% | 3% |
| Polyoxyethylene oleyl ether *4 | 1% | 1% | 1% | 1% | 1% | 1% | 1% |
| Glycerin *5 | 5% | 5% | 5% | 5% | 5% | 5% | 5% |
| Purified water | Balance | Balance | Balance | Balance | Balance | Balance | Balance |
| Total | 100% | 100% | 100% | 100% | 100% | 100% | 100% |
| pH | 5.6 | 5.7 | 5.5 | 5.5 | 5.6 | 5.7 | 8.2 |
| Frizziness improvement rate | 11% | 9% | 10% | 11% | 12% | 4% | 9% |
| Lowering rate of breaking strength | 6% | 7% | 5% | 5% | 6% | 6% | 13% |

*1: LION CORPORATION Arquad T-30
*2: KANTO CHEMICAL CO., INC.
*3: Toray Dow Corning Silicone SF8457
*4: Nihon Emulsion Co., Ltd. EMALEX510
*5: KANTO CHEMICAL CO., INC. Special grade

### Example 30

### Preparation of Hair Lotions

Hair lotions were prepared by the procedures described below according to the compositions shown in Table 5.

Propylene glycol and polyoxyethylene stearyl ether were added to ethyl alcohol to give a solution. Polyvinyl pyrrolidone was added to the solution and was thereby wetted. Thereafter, 65 g of purified water was gradually added with stirring, and the pH of the liquid was adjusted by addition of disodium hydrogen phosphate and sodium dihydrogen phosphate with stirring. 2-Ethoxyethyl thioglycolate obtained in Synthetic Example 2 was added to the pH-adjusted liquid, and the mixture was stirred sufficiently. Thereafter, the pH was readjusted, and purified water was added so that the amount of the pH-adjusted hair lotion became 100 g, followed by stirring.

### [Measurement of Frizz Relaxing Effect]

One end of the frizzy hair sample was clasped with a clip, and the clip was tied to a holding support to suspend the frizzy hair sample. The hair lotion was sprayed to the suspended hair using a hand sprayer, so that the hair was evenly wet. A weight weighing approximately 5 g was attached to the other end of the suspended hair sample, and the sample was allowed to stand at 30° C. for 10 minutes, followed by removing the weight and air-drying.

Thereafter, the dry frizzy hair was measured for posttreatment frizziness in a manner similar to that used for the shampoo treatment evaluation. Separately, the hair was treated with the hair lotion repeatedly ten times, and the hair break was tested in a manner similar to that used for the shampoo treatment evaluation.

The results are shown in Table 5.

### Example 31

The procedures in Example 30 were repeated except that the mercapto compound was replaced with 2-ethoxyethyl thiolactate obtained in Synthetic Example 3 and the composition was altered as shown in Table 5. The results are shown in Table 5.

### Example 32

The procedures in Example 30 were repeated except that the mercapto compound was replaced with 2-mercapto-4-butanolide obtained in Synthetic Example 5 and the composition was altered as shown in Table 5. The results are shown in Table 5.

### Comparative Examples 6 and 7

The procedures in Example 30 were repeated except that the mercapto compound was replaced with sodium sulfite and the composition was altered as shown in Table 5. The results are shown in Table 5.

TABLE 5
Hair lotion

| Ingredients | Ex. 30 | Ex. 31 | Ex. 32 | Comp. Ex. 6 | Comp. Ex. 7 |
|---|---|---|---|---|---|
| 2-Ethoxyethyl thioglycolate | 1.3% | — | — | — | — |
| 2-Ethoxyethyl thiolactate | — | 1.5% | — | — | — |
| 2-Mercapto-4-butanolide | — | — | 1.0% | — | — |
| Sodium sulfite | — | — | — | 1.1% | 1.1% |
| Polyvinyl pyrrolidone *1 | 3% | 3% | 3% | 3% | 3% |
| Propylene glycol *2 | 2% | 2% | 2% | 2% | 2% |
| Polyoxyethylene stearyl ether *3 | 1.5% | 1.5% | 1.5% | 1.5% | 1.5% |
| Ethyl alcohol *4 | 10% | 10% | 10% | 10% | 10% |
| Purified water | Balance | Balance | Balance | Balance | Balance |
| Total | 100% | 100% | 100% | 100% | 100% |
| pH | 6.1 | 6.1 | 6.2 | 6.2 | 8.3 |
| Frizziness improvement rate | 6% | 7% | 5% | 1% | 8% |
| Lowering rate of breaking strength | 4% | 3% | 3% | 2% | 4% |

*1: ISP JAPAN POVIDERM SK3
*2: KANTO CHEMICAL CO., INC.
*3: Nihon Emulsion Co., Ltd. EMALEX620
*4: KANTO CHEMICAL CO., INC. Special grade

The above results prove that the hair relaxers containing the mercapto compounds according to the present invention possess high effects of shaping and relaxing hair in a wide range of pH levels from weak acidity to weak alkalinity. Furthermore, the results establish that the hair relaxers produce increased effects of shaping and relaxing hair in a weakly acidic to neutral pH range, and consequently the damage to hair is minimal.

1. A hair relaxer comprising at least one mercapto compound represented by the following formula (1) or (2): 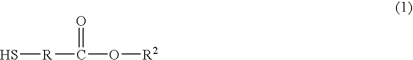
wherein R is an alkylene group of 1 to 6 total carbon atoms that may have a branch; R² is an alkoxyalkyl group of 3 to 15 total carbon atoms; and an alkylene part in R² may contain an ether linkage; 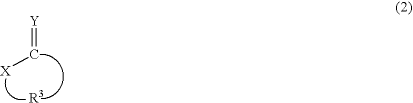
wherein X is a structure selected from the group consisting of a single bond, —O—, —S—, —NH— and —NR⁴—; R⁴ is an alkyl group of 1 to 6 carbon atoms; Y is an oxygen atom or a sulfur atom; and R³ is a divalent organic residue having at least one mercapto group.

2. The hair relaxer according to claim 1, wherein the mercapto compound represented by the formula (1) is a compound represented by the following formula (1a): 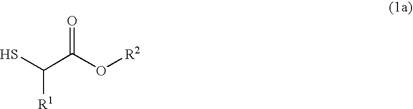
wherein R¹ is a hydrogen atom or an alkyl group of 1 to 5 carbon atoms; and R² is as defined in the formula (1).

3. The hair relaxer according to claim 2, wherein R¹ in the formula (1a) is a hydrogen atom or a methyl group.

4. The hair relaxer according to claim 2, wherein R² in the formula (1a) is a group selected from the group consisting of 2-methoxyethyl, 2-ethoxyethyl, 2-methoxypropyl, 2-ethoxypropyl, 1-methoxypropane-2-yl, 1-ethoxypropane-2-yl, 5-methoxy-3-oxapentyl and 5-ethoxy-3-oxapentyl.

5. The hair relaxer according to claim 2, wherein the mercapto compound represented by the formula (1a) is a compound selected from the group consisting of 2-methoxyethyl thioglycolate, 2-ethoxyethyl thioglycolate, 2-methoxyethyl thiolactate and 2-ethoxyethyl thiolactate.

6. The hair relaxer according to claim 1, wherein the mercapto compound represented by the formula (1) is a compound represented by the following formula (1b): 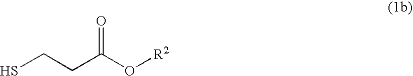
wherein R² is as defined in the formula (1).

7. The hair relaxer according to claim 6, wherein R² in the formula (1b) is a group selected from the group consisting of 2-methoxyethyl, 2-ethoxyethyl, 2-methoxypropyl, 2-ethoxypropyl, 1-methoxypropane-2-yl, 1-ethoxypropane-2-yl, 5-methoxy-3-oxapentyl and 5-ethoxy-3-oxapentyl.

8. The hair relaxer according to claim 6, wherein the mercapto compound represented by the formula (1b) is a compound selected from the group consisting of 2-methoxyethyl 3-mercaptopropionate and 2-ethoxyethyl 3-mercaptopropionate.

9. The hair relaxer according to claim 1, wherein X in the formula (2) is a structure selected from the group consisting of —O—, —NH—, —S— and —N(CH₃)—.

10. The hair relaxer according to claim 1, wherein X in the formula (2) is a single bond.

11. The hair relaxer according to claim 1, wherein Y in the formula (2) is an oxygen atom.

12. The hair relaxer according to claim 1, wherein R³ in the formula (2) is an alkylene group having at least one mercapto group.

13. The hair relaxer according to claim 1, wherein when Y and R³ in the formula (2) are an oxygen atom and an alkylene group having at least one mercapto group, respectively, the mercapto group of R³ is bonded at the α-position of the carbonyl group.

14. The hair relaxer according to claim 1, wherein the mercapto compound represented by the formula (2) is a compound selected from the group consisting of 2-mercapto-4-butanolide, 2-mercapto-4-methyl-4-butanolide, 2-mercapto-4-ethyl-4-butanolide, 2-mercapto-4-butyrolactam, N-methyl-2-mercapto-4-butyrolactam, 2-mercapto-5-valerolactam, N-methyl-2-mercapto-5-valerolactam and 2-mercapto-6-hexanolactam.

15. The hair relaxer according to claim 1, wherein the mercapto compound represented by the formula (2) is a compound selected from the group consisting of 2-mercaptocyclopentanone and 2-mercaptocyclohexanone.

16. The hair relaxer according to claim 1, wherein the hair relaxer contains the mercapto compound in an amount of 0.1 to 10% by mass.

17. The hair relaxer according to claim 1, wherein the pH is in the range of 4.0 to 7.5.

18. A shampoo comprising the hair relaxer as claimed in claim 1.

19. A rinsing conditioner comprising the hair relaxer as claimed in claim 1.

20. A conditioner comprising the hair relaxer as claimed in claim 1.

21. A hair treatment comprising the hair relaxer as claimed in claim 1.

22. A hair lotion comprising the hair relaxer as claimed in claim 1.

23. A hair mousse comprising the hair relaxer as claimed in claim 1.
